(19) United States
(12) Patent Application Publication
(10) Pub. No.: US 2005/0004029 A1
(43) Pub. Date: Jan. 6, 2005

(54) Methods and compositions for treating vascular leak using hepatocyte growth factor

(76) Inventor: Joe G.N. Garcia, Pasadena, MD (US)

Correspondence Address: EDWARDS & ANGELL, LLP, P.O. BOX 55874, BOSTON, MA, 02205, US

(21) Appl. No.: 10/836,320

(22) Filed: Apr. 30, 2004

Related U.S. Application Data
(63) Continuation of application No. PCT/US02/34968, filed on Nov. 1, 2002.
(60) Provisional application No. 60/335,341, filed on Nov. 1, 2001.

(51) Int. Cl. A61K 38/18

(52) U.S. Cl. 514/12

(57) ABSTRACT

News methods are provided for treating vascular leak, including acute lung injury. Therapies of the invention include administration of hepatocyte growth factor to a subject in need thereof, such as a subject suffering from or susceptible to pneumonia or sepsis, or chronic conditions that can result from vascular leak.

### RELATED APPLICATIONS

This application claims the benefit of U.S. Provisional Application Ser. No. 60/335,341, filed Nov. 1, 2001, the entire contents of which are hereby incorporated by this reference.

### GOVERNMENT SUPPORT

This invention resulted from research funded in whole or in part by the National Institutes of Health, Grant Nos. HL-58064, HL-50533, and HL-03666. The Federal Government may have certain rights in this invention.

### BACKGROUND OF THE INVENTION

Hepatocyte growth factor (HGF), also known as scatter factor, is a heparin-binding glycoprotein originally identified as a fibroblast product that induces scattering of contiguous epithelium sheets into isolated cells (Stoker, M. et al. (1987) Nature 327(6119):239-242). Subsequently, HGF was recognized as a multifunctional cytokine secreted by several cell types (Rosen, E. M. and Goldberg, I. D. (1995) Adv. Cancer Res. 67:57-279) displaying diverse biological effects including mitogenesis, motogenesis, morphogenesis, organogenesis, and cell survival (Matsumoto, K. and Nakamura, T. (1996) J. Biochem. (Tokyo) 119(4):591-600; Zhang, L. et al. (2000) J. Neurosci. Res. 59(4):489-496). More recently, HGF was noted to elicit potent angiogenic activities (Bussolino, F. et al. (1992) J. Cell Biol. 119(3):629-641; Grant, D. S. et al. (1993) Proc. Natl. Acad. Sci. USA 90:1937-1941) mediated primarily through direct endothelial cell stimulation of cell motility, proliferation, protease production, invasion, and organization into capillary-like tubes (Rosen, E. M. and Goldberg, I. D. (1995) Adv. Cancer Res. 67:57-279). These complex biological functions occur via ligation of the HGF tyrosine kinase receptor known as c-Met, which is composed of a 50-kDa extracellular α-subunit and a 145-kDa transmembrane β-subunit (Bottaro, D. P. et al. (1991) Science 251:802-804). The β-subunit of c-Met contains tyrosine kinase domains, tyrosine phosphorylation sites, and tyrosine docking sites (Nguyen, L. et al. (1997) J. Biol. Chem. 272:20811-20819). Binding of HGF with the receptor stimulates receptor tyrosine kinase activity, leading to autophosphorylation of the receptor, followed by the recruitment of multiple SH2 domain containing signaling molecules, including Gab1, Grb2, phosphatidylinositol 3′ kinase (PI-3′ kinase), PLC-γ, p60src, Shc, and Shp2 (Ponzetto, C. et al. (1994) Cell 77:261-271; Schaeper, U. et al. (2000) J. Cell Biol. 149(7):1419-1432), signaling components likely involved in diverse responses which include the prevention of apoptosis (Holgado-Madruga, M. et al. (1997) Proc. Natl. Acad. Sci. USA 94(23):12419-12424), activation of mitogen-activated protein kinase (MAPK) pathways, and branching morphogenesis (Schaeper, U. et al. (2000) J. Cell Biol. 149(7):1419-1432).

The complex angiogenic effects of HGF have not been studied in the pulmonary circulation where the pulmonary vascular endothelium function as a semi-selective barrier regulating the exchange of fluid, macromolecules, and cells between blood vessels and the surrounding lung tissues. Vascular barrier regulation is also involved in the multi-faceted process of angiogenesis (Garcia, J. G. N. et al. (2001) J. Clin. Invest. 108:689-711; Thurston, G. et al. (1999) Science 286:2511-2514), as newly formed capillaries are leaky and therefore not fully functional (Thurston, G. et al. (2000) Nat. Med. 6:460-463). Several angiogenic factors regulate vascular barrier function including vascular endothelial growth factor (VEGF), formerly known as vascular permeability factor (Connolly, D. T. et al. (1989) J. Clin. Invest. 84(5):1470-1478), and angiopoietin-1 and -2 (Thurston, G. et al. (2000) Nat. Med. 6:460-463). Increases in VEGF are observed in inflammatory lung syndromes (Connolly, D. T. et al. (1989) J. Clin. Invest. 84(5):1470-1478), in the ischemic lung (Becker, P. M. et al. (2001) Am. J. Physio. Lung Cell. Mol. Physiol. 281(6):L1500-11), and may contribute to endothelial cell activation, formation of intercellular gaps and the increased vascular permeability and life-threatening edema (Dudek, S. M. and Garcia, J. G. N. (2001) J. Appl. Physiol. 91:1487-1500) in patients with acute lung injuries. The platelet phospholipid growth factor sphingosine 1-phosphate (Garcia, J. G. N. et al. (2001) J. Clin. Invest. 108:689-711) was previously identified as a complete angiogenic factor, and its participation has been described in the terminal angiogenic effect characterized by barrier stabilization of the newly formed but leaky vessels (Carmeliet, P. (2000) Nat. Med. 6(4):389-395) and in cultured human endothelium via ligation of Edg-1 receptors (Garcia, J. G. N. et al. (2001) J. Clin. Invest. 108:689-711). Targeted disruption of the Edg-1 gene in mice leads to embryonic lethality with progressive edema formation and hemorrhage (Liu, Y. et al. (2000) J. Clin. Invest. 106:951-961). Thus, the maintenance of the normal endothelial cell barrier and the integrity of the microcirculation is also a final event of new blood vessel formation.

### SUMMARY OF THE INVENTION

The present invention is based, at least in part, on the discovery that hepatocyte growth factor, also referred to interchangeably herein as “HGF,” plays a role in the regulation of human pulmonary vascular endothelial barrier integrity. The present invention is further based, at least in part, on the identification of signaling pathways which mediate HGF-evoked barrier alterations. The discoveries of the present invention demonstrate that HGF potently enhances endothelial cell barrier integrity, i.e. reduces permeability as determined by increases in transendothelial electrical resistance. These changes occur in association with increased cortical actin rearrangement, and improved adherens junction integrity as determined by VE-cadherin/β-catenin association with the cytoskeleton. Both physiologic and immunofluorescent events are dependent upon phosphatidylinositol 3-kinase (PI-3′ K), mitogen-activated protein kinase, and protein kinase C activity. Accordingly, the present invention provides therapies for treatment against vascular leak.

Methods of the invention include treatment of mammalian cells, particularly primate cells, especially human cells, with HGF or functional derivative thereof, that can modulate vascular barrier integrity and/or endothelial permeability, particularly compounds that can positively impact vascular barrier integrity and decrease endothelial permeability, i.e., decrease vascular leak.

Methods of the invention particularly include treating cells that have been subjected to vascular leak, particularly acute lung injury. Lung endothelial cells are particularly preferred. For example, a subject suffering from or susceptible to acute lung injury (e.g., pneumonia or sepsis) can be treated in accordance with the invention.

Treatment methods of the invention include administration to a mammal in need of such treatment a therapeutically effective amount of HGF or functional derivative thereof that can positively impact vascular barrier integrity and decrease endothelial permeability in an animal, including a mammal, particularly a human. Preferably, a subject is identified and selected that is susceptible to or suffering from a condition associated with, caused by, or related to vascular leak, e.g., acute lung injury such as that associated with pneumonia, sepsis, trauma, inflammation, infection, pulmonary aspiration of stomach contents, pulmonary aspiration of water, near drowning, burns, inhalation of noxious fumes, fat embolism, blood transfusion, amniotic fluid embolism, air embolism, preeclampsia, eclampsia, vascular leak syndrome, edema, organ failure, poisoning, and/or radiation. In some embodiments, the HGF or functional derivative thereof is administered in combination with an effective amount of sphingosine 1-phosphate. In other embodiment, the HGF or functional derivative thereof is administered about 6 hours, about 18 hours, or about 1 week after acute lung injury. In preferred embodiments, the HGF or functional derivative thereof is administered intravenously or via bronchial injection.

Other aspects of the invention are disclosed infra.

### BRIEF DESCRIPTION OF THE DRAWINGS

FIGS. 1A-1D depict HGF-mediated increases in human transendothelial electrical resistance (TER). FIG. 1A: Human pulmonary artery endothelial cells were grown to confluence on gelatinized gold microelectrodes. Two hours prior to TER measurement, growth medium was replaced with serum-free M199. Serial-diluted HGF was added to cells at indicated concentrations and TER monitored for 2.5 hr. HGF dose-dependently increased TER consistent with barrier enhancement. The result shown is a representative TER tracing of three independent experiments. FIG. 1B: Similar to FIG. 1A, human alveolar epithelial cells (A549) were grown on gold microelectrodes and challenged with vehicle, HGF, (100 ng/mL) or sphingosine 1-phosphate (1 μM). Depicted is the differential sensitivity to sphingosine 1-Phosphate, whereas HGF was completely without effect. These results indicate that HGF increases in electrical resistance and enhanced paracellular integrity are specific to endothelium. FIG. 1C: In these experiments, HGF (1, 10 and 100 ng/ml) was added to human endothelial monolayers prior to subsequent re-stimulation at 2 hr with HGF (10 ng/ml). Whereas the HGF barrier-protective response was not altered by prior HGF stimulation at 1 ng/ml, pretreatment with HGF at 10 and 100 ng/mL significantly reduced the subsequent HGF responses, consistent with receptor desensitization. FIG. 1D: NK2 is a truncated form of HGF which in some cell systems reproduces the full HGF effect. The addition of up to 100 ng/ml of NK2 failed to directly alter TER and did not influence subsequent HGF-mediated increases in TER.

FIGS. 2A-2B depict the effect of PI-3′ kinase inhibition on HGF-induced endothelial cell cortical actin rearrangement and barrier enhancement. FIG. 2A: Human pulmonary artery endothelial monolayers were pretreated with LY294002 (25 μM, 1 hr) or vehicle control, followed by stimulation with HGF (20 ng/ml). TER was monitored for 2.5 hr. The maximal increases in TER elicited by HGF were expressed as the percentage increase over vehicle control (data collected at 15 min after HGF addition). The reductions of HGF-induced TER increases by LY294002 were expressed as a percentage of the maximal TER increases by HGF in the absence of the inhibitor. LY294002 significantly attenuated increases in TER stimulated by HGF. Data represent mean±SD from three independent experiments (two wells each). FIG. 2B: The electrical resistance tracing is a representative experiment (n=3) demonstrating the effect PI-3′ kinase inhibition by LY294002 on the increase in TER induced by HGF. Data is presented as normalized resistance.

FIGS. 3A-3C depict the effect of MAPK inhibitors on the increases in TER induced by HGF. FIG. 3A: Human pulmonary artery endothelial monolayers were pretreated with the ERK kinase (MEK) inhibitor UO126 (10 μM, 1 hr), the p38 inhibitor SB203580 (20 μM, 1 hr), or vehicle control, followed by stimulation with HGF (20 ng/ml). TER was continuously monitored for 2.5 hr. UO126 and SB203580 significantly blocked HGF-induced increases in TER. Data are mean±SEM, n=3 for the UO126 experiment, n=4 for the SB203580 experiment. FIG. 3B: Depicted is the HGF-mediated TER response in the presence and absence of p38 MAP kinase inhibition with SB203580. Inhibition of p38 MAP kinase produces marked reduction in the HGF-mediated increases in TER. FIG. 3C: Similar to the experiments in FIG. 5A, human endothelial cells were exposed to a combination of LY294002 (25 μM) and UO126 (10 μM), which produced near total abolishment of the HGF-mediated increase in TER.

FIGS. 4A-4B depict the involvement of PKC activities in HGF-induced barrier enhancement. FIG. 4A: Endothelial monolayers grown on gold microelectrodes were pretreated with the specific pan PKC inhibitor Ro-31-2880 (10 μM, 1 hr) or vehicle control, followed by stimulation with HGF (20 ng/ml). TER was continuously monitored for 2.5 hr. Ro-31-2880 significantly attenuated HGF-induced increases in TER. Data represent mean±SD from four independent experiments. FIG. 4B: The effect of Ro-31-2880 on HGF-mediated increases in TER is depicted. PKC inhibition produced significant elevation in TER alone but blunted the HGF response.

### DETAILED DESCRIPTION OF THE INVENTION

The present invention is based, at least in part, on the discovery that hepatocyte growth factor, also referred to interchangeably herein as “HGF,” plays a role in the regulation of human pulmonary vascular endothelial barrier integrity. The present invention is further based, at least in part, on the identification of signaling pathways which mediate HGF-evoked barrier alterations. The discoveries of the present invention demonstrate that HGF can potently enhance endothelial cell barrier integrity, i.e. can reduce permeability as determined by increases in transendothelial electrical resistance. These changes occur in association with increased cortical actin rearrangement, and improved adherens junction integrity as determined by VE-cadherin/β-catenin association with the cytoskeleton. Both physiologic and immunofluorescent events are dependent upon phosphatidylinositol 3-kinase (PI-3′ K), mitogen-activated protein kinase, and protein kinase C activity.

The stabilization of endothelial cell (EC) barrier function within newly formed capillaries is a critical feature of angiogenesis. The results presented herein examined human lung EC barrier regulation elicited by hepatocyte growth factor (HGF), a recognized angiogenic factor and EC chemoattractant. HGF rapidly and dose-dependently elevated transendothelial electrical resistance (TER) of EC monolayers (>50% increase at 100 ng/ml) with immunofluorescent microscopic evidence of both cytoplasmic actin stress fiber dissolution and strong augmentation of the cortical actin ring. HGF rapidly stimulated phosphoinositide 3′ kinase (PI-3′ kinase), ERK1/2, p38 MAP kinase and protein kinase C (PKC) activities, and pharmacologic inhibitor studies demonstrated each pathway to be intimately involved in HGF-induced increases in TER and cortical actin thickening. The results presented herein also examined whether the Ser/Thr glycogen synthase kinase 3β (GSK3β) represents a potential target for the HGF barrier-promoting response. HGF induced significant GSK3β phosphorylation which was attenuated by inhibition of PI-3′ kinase, MEK, p38 MAPK, and membrane-associated PKC activities, and strongly correlated with reductions in both HGF-induced TER as well as enhanced β-catenin immunoreactivity observed at cell-cell junctions. The results herein suggest a model where HGF-mediated EC cytoskeletal rearrangement and barrier enhancement are critically dependent upon the activation of a complex kinase cascade which converges at GSK3β to increase the availability of p catenin thereby enhancing endothelial junctional integrity and vascular barrier function, and permeability to water and solute are likely diminished. Accordingly, the invention provides methods to use HGF to treat acute lung injury.

Methods of Treatment

As stated above, and demonstrated in the examples which follow, it has now been found that administration of HGF can be effective to treat against or inhibit vascular leak, including vascular leak induced by acute lung injury.

Therapeutic methods of the invention include selecting or identifying mammalian cells or a mammalian subject that that is suffering from or susceptible to vascular leak, particularly as a result of acute lung injury and administering to the cells or subject effective amounts of HGF or functional derivative thereof. Exemplary cells for treatment include various eukaryotic cells e.g. lung epithelial cells.

Typical subjects for treatment include mammals suffering from or susceptible to acute lung injury. The term “acute lung injury”, as used herein, is a disorder or syndrome characterized by hypoxemic respiratory failure, as defined by Bernard, G. R. et al. (1994) Am. J. Respir. Crit. Care Med. 149(3 Pt 1):818-824. A severe form of acute lung injury is referred to as “Acute Respiratory Distress Syndrome”. Acute lung injury may also be characterized by airway collapse (low lung volumes), surfactant deficiency and/or reduced lung compliance.

The methods of the invention are useful for treating vascular leak caused by, associated with, or related to acute lung injury, particularly pneumonia or sepsis, or other event involving vascular leak, including, but not limited to, trauma, inflammation, infection, pulmonary aspiration of stomach contents, pulmonary aspiration of water, near drowning, burns, inhalation of noxious fumes, fat embolism, blood transfusion, amniotic fluid embolism, air embolism, preeclampsia, eclampsia, vascular leak syndrome, edema, organ failure, poisoning, and/or radiation.

Preferred compounds for use in therapeutic methods of the invention are HGF proteins or functional derivatives thereof. Preferred compounds have an HGF activity, including one or more of the following activities: (1) binding and/or activation of the c-met receptor; (2) activation of PI-3′ kinase activity; (3) induction of Akt phosphorylation; (4) activation of mitogen-activated protein kinase activity; (5) activation of protein kinase C activity; (6) induction of GSK-3β phosphorylation; (6) induction of β-catenin localization to the cortical cytoskeleton; (7) increasing of transendothelial electrical resistance (TER); (8) enhancing of vascular barrier integrity; and (9) decreasing of vascular leak.

In particular, suitable assays for determining whether an HGF derivative has HGF activity are disclosed herein. Preferably, the HGF or functional derivative thereof will increase vascular barrier integrity (e.g. as assessed by TER) by a detectable amount relative to a control in a TER assay as set forth in the Examples presented herein. In particular, preferably treatment with HGF or functional derivative thereof increases TER by at least about 10%, 20%, 30%, 40%, 50%, 60%, 70%, 80%, 90% or 100% in a TER assay relative to a control (i.e. the same assay where the cells have not been exposed to HGF or functional derivative thereof).

As discussed above, the invention includes methods for treating preventing certain vascular leak disorders, including the consequences of pneumonia and sepsis comprising the administration of an effective amount of HGF or functional derivative thereof to a subject including a mammal, such as a primate, especially a human, in need of such treatment. In particular, the invention provides methods for treatment and/or prophylaxis of vascular leak, e.g., vascular leak resulting from acute lung injury such as pneumonia or sepsis. The methods of the invention are also useful for treating other disorders, diseases, and/or conditions associated with, caused by, or related to vascular leak, including, but not limited to: trauma, inflammation, infection, pulmonary aspiration of stomach contents, pulmonary aspiration of water, near drowning, burns, inhalation of noxious fumes, fat embolism, blood transfusion, amniotic fluid embolism, air embolism, preeclampsia, eclampsia, vascular leak syndrome, edema, organ failure, poisoning, and/or radiation. Reduction of vascular leak in the lungs using the methods of the invention may also lead to reduction of vascular leak in other tissues. Therefore, the methods of the invention may be useful in treating vascular leak in any tissue, organ, or area of the body.

Compounds for use in the methods of the invention can be administered intranasally, orally or by injection, e.g., intramuscular, intraperitoneal, subcutaneous or intravenous injection, or by transdermal, intraocular or enteral means. The optimal dose can be determined by conventional means. In a preferred embodiment, HGF or functional derivative thereof is administered intravenously. In another embodiment, HGF or functional derivative thereof is administered by bronchoscopic injection, or by other standard means for applying compounds directly to the lungs, for example, using an inhaled aerosol. Compounds for use in the methods of the invention are suitably administered to a subject in the protonated and water-soluble form, e.g., as a pharmaceutically acceptable salt of an organic or inorganic acid, e.g., hydrochloride, sulfate, hemi-sulfate, phosphate, nitrate, acetate, oxalate, citrate, maleate, mesylate, etc.

Compounds for use in the methods of the invention can be employed, either alone or in combination with one or more other therapeutic agents as discussed above, as a pharmaceutical composition in mixture with conventional excipient, i.e., pharmaceutically acceptable organic or inorganic carrier substances suitable for parenteral, enteral or intranasal application which do not deleteriously react with the active compounds and are not deleterious to the recipient thereof. Suitable pharmaceutically acceptable carriers include but are not limited to water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethylcellulose, polyvinylpyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds.

For parenteral application, particularly suitable are solutions, preferably oily or aqueous solutions as well as suspensions, emulsions, or implants, including suppositories. Ampules are convenient unit dosages.

For enteral application, particularly suitable are tablets, dragees or capsules having talc and/or carbohydrate carrier binder or the like, the carrier preferably being lactose and/or corn starch and/or potato starch. A syrup, elixir or the like can be used wherein a sweetened vehicle is employed. Sustained release compositions can be formulated including those wherein the active component is protected with differentially degradable coatings, e.g., by microencapsulation, multiple coatings, etc.

For topical applications, formulations may be prepared in a topical ointment or cream containing one or more compounds of the invention. When formulated as an ointment, one or more compounds of the invention suitably may be employed with either a paraffinic or a water-miscible base. The one or more compounds also may be formulated with an oil-in-water cream base. Other suitable topical formulations include e.g. lozenges and dermal patches.

Intravenous or parenteral administration, e.g., sub-cutaneous, intraperitoneal or intramuscular administration are generally preferred.

For in vitro applications, a multi-well plate or other reaction substrate may be suitably employed.

It will be appreciated that the actual preferred amounts of active compounds used in a given therapy will vary according to the specific compound being utilized, the particular compositions formulated, the mode of application, the particular site of administration, etc. Optimal administration rates for a given protocol of administration can be readily ascertained by those skilled in the art using conventional dosage determination tests conducted with regard to the foregoing guidelines. In general, a suitable effective dose of one or more compounds of the invention, particularly when using the more potent compound(s) of the invention, will be in the range of from 0.01 to 100 milligrams per kilogram of bodyweight of recipient per day, preferably in the range of from 0.01 to 20 milligrams per kilogram bodyweight of recipient per day, more preferably in the range of 0.05 to 4 milligrams per kilogram bodyweight of recipient per day. The desired dose is suitably administered once daily, or several sub-doses, e.g. 2 to 4 sub-doses, are administered at appropriate intervals through the day, or other appropriate schedule.

Peptides and Peptidomimetics

The wild type HGF amino acid and nucleic acid sequences are disclosed in GenBank Accession Nos. XP_{—}168542 and XM_{—}168542, respectively. The invention utilizes proteins, derivatives of proteins (including peptides and peptide fragments), and compositions which are proteins or derivatives of proteins linked to a coupling partner.

As is well understood, identity at the amino acid level is generally defined and determined by the TBLASTN program, of Altschul et al, J. Mol. Biol., 215:403-10, 1990, which is in standard use in the art. Sequence identity may be over the full-length of the relevant peptide or over a contiguous sequence of about 5, 10, 15, 20, 25, 30 or 35 amino acids, compared with the relevant wild-type amino acid sequence. Preferably, the amino acid sequence of the peptides used in the methods of the invention share at least 75%, or 80%, or 85% identity, and more preferably at least 90% or 95% identity sequence identity with the corresponding part of the full length human HGF sequences.

The present invention also provides sequence variants of the above peptides. In one embodiment, the variants are peptide fragments of HGF including 1, 2, 3, 4, 5, greater than 5, or greater than 10 amino acid alterations such as substitutions, deletions or insertions with respect to the wild-type sequence.

Peptide or protein derivatives of the peptides or proteins and sequence variants described above include pharmaceutically acceptable salts of the peptides or proteins, alkyl esters, amides, alkylamides, dialkylamides, wherein the alkyl groups are preferably lower alkyl such as C1-4.

The present invention further includes provides peptides or proteins which are composed of D and L amino acids, or combinations thereof. Alternatively or additionally, the proteins, peptides, variants and derivatives may be part of a larger peptide, which may or may not include an additional portion of HGF, e.g. 1, 2, 3, 4, 5 or 10 or more additional amino acids, adjacent to the relevant specific peptide fragment in HGF, or heterologous thereto may be included at one end or both ends of the protein or peptide.

Coupling partners

The invention also includes derivatives of the peptides, including the peptide linked to a coupling partner, e.g. an effector molecule, an immunogen, a label, a drug, a toxin and/or a carrier or transport molecule. Techniques for coupling the peptides used in the methods of the invention to both peptidyl and non-peptidyl coupling partners are well known in the art. In one embodiment, the carrier molecule is a 16 aa peptide sequence derived from the homeodomain of Antennapedia (e.g. as sold under the name “Penetratin”), which can be coupled to a peptide via a terminal Cys residue. The “Penetratin” molecule and its properties are described in WO 91/18981.

Synthesis

Peptides may be generated wholly or partly by chemical synthesis. The compounds of the present invention can be readily prepared according to well-established, standard liquid or, preferably, solid-phase peptide synthesis methods, general descriptions of which are broadly available (see, for example, in J. M. Stewart and J. D. Young, Solid Phase Peptide Synthesis, 2nd edition, Pierce Chemical Company, Rockford, Ill. (1984), in M. Bodanzsky and A. Bodanzsky, The Practice of Peptide Synthesis, Springer Verlag, New York (1984); and Applied Biosystems 430A Users Manual, ABI Inc., Foster City, Calif.), or they may be prepared in solution, by the liquid phase method or by any combination of solid-phase, liquid phase and solution chemistry, e.g. by first completing the respective peptide portion and then, if desired and appropriate, after removal of any protecting groups being present, by introduction of the residue X by reaction of the respective carbonic or sulfonic acid or a reactive derivative thereof.

Expression

Another convenient way of producing a peptidyl molecule according to the present invention (peptide or polypeptide) is to express nucleic acid encoding it, by use of nucleic acid in an expression system. Accordingly the present invention also provides in various aspects nucleic acid encoding the polypeptides and peptides of the invention.

Generally, nucleic acid according to the present invention is provided as an isolate, in isolated and/or purified form, or free or substantially free of material with which it is naturally associated, such as free or substantially free of nucleic acid flanking the gene in the human genome, except possibly one or more regulatory sequence(s) for expression.

Nucleic acid may be wholly or partially synthetic and may include genomic DNA, cDNA or RNA. Where nucleic acid according to the invention includes RNA, reference to the sequence shown should be construed as reference to the RNA equivalent, with U substituted for T.

Nucleic acid sequences encoding a polypeptide or peptide in accordance with the present invention can be readily prepared by the skilled person using the information and references contained herein and techniques known in the art (for example, see Sambrook, Fritsch and Maniatis, “Molecular Cloning, A Laboratory Manual, Cold Spring Harbor Laboratory Press, 1989, and Ausubel et al, Short Protocols in Molecular Biology, John Wiley and Sons, 1992), given the nucleic acid sequence and clones available. These techniques include (i) the use of the polymerase chain reaction (PCR) to amplify samples of such nucleic acid, e.g. from genomic sources, (ii) chemical synthesis, or (iii) preparing cDNA sequences. DNA encoding HGF fragments may be generated and used in any suitable way known to those of skill in the art, including by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Modifications to the HGF sequences can be made, e.g. using site directed mutagenesis, to lead to the expression of modified HGF peptide or to take account of codon preference in the host cells used to express the nucleic acid.

In order to obtain expression of the nucleic acid sequences, the sequences can be incorporated in a vector having one or more control sequences operably linked to the nucleic acid to control its expression. The vectors may include other sequences such as promoters or enhancers to drive the expression of the inserted nucleic acid, nucleic acid sequences so that the polypeptide or peptide is produced as a fusion and/or nucleic acid encoding secretion signals so that the polypeptide produced in the host cell is secreted from the cell. Polypeptide can then be obtained by transforming the vectors into host cells in which the vector is functional, culturing the host cells so that the polypeptide is produced and recovering the polypeptide from the host cells or the surrounding medium. Prokaryotic and eukaryotic cells are used for this purpose in the art, including strains of E. coli, yeast, and eukaryotic cells such as COS or CHO cells.

Accordingly, the present invention also encompasses a method of making a polypeptide or peptide, the method including expression from nucleic acid encoding the polypeptide or peptide. This may conveniently be achieved by growing a host cell in culture, containing such a vector, under appropriate conditions which cause or allow expression of the polypeptide. Polypeptides and peptides may also be expressed in in vitro systems, such as reticulocyte lysate.

Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 1992.

Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary cells, HeLa cells, baby hamster kidney cells, COS cells, U-2-OS cells, SAOS-2 cells and many others. A common, preferred bacterial host is E. coli.

Thus, a further aspect of the present invention provides a host cell containing heterologous nucleic acid as disclosed herein.

The nucleic acid of the invention may be integrated into the genome (e.g. chromosome) of the host cell. Integration may be promoted by inclusion of sequences which promote recombination with the genome, in accordance with standard techniques. The nucleic acid may be on an extra-chromosomal vector within the cell, or otherwise identifiably heterologous or foreign to the cell.

A still further aspect provides a method which includes introducing the nucleic acid into a host cell. The introduction, which may (particularly for in vitro introduction) be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. As an alternative, direct injection of the nucleic acid could be employed.

Marker genes such as antibiotic resistance or sensitivity genes may be used in identifying clones containing nucleic acid of interest, as is well known in the art.

The introduction may be followed by causing or allowing expression from the nucleic acid, e.g. by culturing host cells (which may include cells actually transformed although more likely the cells will be descendants of the transformed cells) under conditions for expression of the gene, so that the encoded polypeptide (or peptide) is produced. If the polypeptide is expressed coupled to an appropriate signal leader peptide it may be secreted from the cell into the culture medium. Following production by expression, a polypeptide or peptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers.

Introduction of nucleic acid encoding a peptidyl molecule according to the present invention may take place in vivo by way of gene therapy, to enhance or promote the interaction between HGF and c-met.

Thus, a host cell containing nucleic acid according to the present invention, e.g. as a result of introduction of the nucleic acid into the cell or into an ancestor of the cell and/or genetic alteration of the sequence endogenous to the cell or ancestor (which introduction or alteration may take place in vivo or ex vivo), may be comprised (e.g. in the soma) within an organism which is an animal, particularly a mammal, which may be human or non-human, such as rabbit, guinea pig, rat, mouse or other rodent, cat, dog, pig, sheep, goat, cattle or horse, or which is a bird, such as a chicken. Genetically modified or transgenic animals or birds comprising such a cell are also provided as further aspects of the present invention.

This procedure may have a therapeutic aim. Also, the presence of a mutant, allele, derivative or variant sequence within cells of an organism, particularly when in place of a homologous endogenous sequence, may allow the organism to be used as a model in testing and/or studying compositions which modulate activity of the encoded polypeptide in vitro or are otherwise indicated to be of therapeutic potential. Conveniently, however, assays for such compositions may be carried out in vitro, within host cells or in cell-free systems.

Suitable screening methods are conventional in the art. They include techniques such as radioimmunosassay, scintillation proximetry assay and ELISA methods. Suitably either the HGF protein or c-met, or a fragment, an analogue, derivative, variant or functional mimetic of any of these protein, is immobilized whereupon the other is applied in the presence of the agents under test. In a scintillation proximetry assay a biotinylated protein fragment is bound to streptavidin coated scintillant-impregnated beads (produced by Amersham). Binding of radiolabeled peptide is then measured by determination of radioactivity induced scintillation as the radioactive peptide binds to the immobilized fragment. Agents which intercept this are thus inhibitors of the interaction.

Alternatively, the phosphorylation of c-met, Akt, or other downstream effectors of HGF signaling, may be measured, such as by incorporation or removal of labeled phosphates, as observed by a signal. Signaling may be observed in a variety of ways known in the art, including radioisotopic, chemical, fluorescent, and enzymatic signaling. Alternatively, the number of mitotic cells in a sample may be measured, such as by flow cytometry, microscopic techniques, visualization, or other techniques known in the art. For example, flow cytometry measurements may involve staining of chromosomes with phospho-histone (H3), a marker of productive entry into mitosis. Screening may be high-throughput or low-throughput.

Mimetic Compounds

Other candidate inhibitor compounds may be based on modeling the 3-dimensional structure of a polypeptide or peptide fragment and using rational drug design to provide potential inhibitor compounds with particular molecular shape, size and charge characteristics.

Following identification of a substance or agent which modulates or affects the activity of HGF, the substance or agent may be investigated further.

As noted, the agent may be peptidyl, e.g., a peptide which includes a sequence as recited above, or may be a functional analogue of such a peptide.

As used herein, the expression “functional analogue” relates to peptide variants or organic compounds having the same functional activity as the peptide in question.

Suitable modeling techniques are known in the art. This includes the design of so-called “mimetics” which involves the study of the functional interactions fluorogenic oligonucleotide the molecules and the design of compounds which contain functional groups arranged in such a manner that they could reproduced those interactions.

The designing of mimetics to a known pharmaceutically active compound is a known approach to the development of pharmaceuticals based on a lead compound. This might be desirable where the active compound is difficult or expensive to synthesize or where it is unsuitable for a particular method of administration, e.g. peptides are not well suited as active agents for oral compositions as they tend to be quickly degraded by proteases in the alimentary canal. Mimetic design, synthesis and testing may be used to avoid randomly screening large number of molecules for a target property.

There are several steps commonly taken in the design of a mimetic from a compound having a given target property. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modeled to according its physical properties, e.g. stereochemistry, bonding, size and/or charge, using data from a range of sources, e.g. spectroscopic techniques, X-ray diffraction data and NMR. Computational analysis, similarity mapping (which models the charge and/or volume of a pharmacophore, rather than the bonding between atoms) and other techniques can be used in this modeling process.

In a variant of this approach, the three-dimensional structure of the ligand and its binding partner are modeled. This can be especially useful where the ligand and/or binding partner change conformation on binding, allowing the model to take account of this in the design of the mimetic.

A template molecule is then selected onto which chemical groups which mimic the pharmacophore can be grafted. The template molecule and the chemical groups grafted on to it can conveniently be selected so that the mimetic is easy to synthesize, is likely to be pharmacologically acceptable, and does not degrade in vivo, while retaining the biological activity of the lead compound. The mimetic or mimetics found by this approach can then be screened to see whether they have the target property, or to what extent they exhibit it. Further optimization or modification can then be carried out to arrive at one or more final mimetics for further testing or optimization, e.g. in vivo or clinical testing.

The mimetic or mimetics found by this approach can then be screened to see whether they have the target property, or to what extent they exhibit it. Further optimization or modification can then be carried out to arrive at one or more final mimetics for in vivo or clinical testing.

Mimetics of this type together with their use in therapy form a further aspect of the invention.

The present invention further provides the use of a peptide which includes a sequence as disclosed, or a derivative, active portion, analogue, variant or mimetic, thereof able to activate c-met, in screening for a composition able to bind HGF and/or having the activity of promoting the binding of HGF to c-met.

Pharmaceutical Uses

The compositions of the invention can be used in the treatment of acute lung injury due to causes such as pneumonia or sepsis. Substances or compositions described in the application can be used individually or in various combinations.

Generally, a composition according to the present invention is provided in an isolated and/or purified form. This may include being in a further composition where it represents at least about 90% active ingredient, more preferably at least about 95%, more preferably at least about 98%. Such a composition may, however, include inert carrier materials or other pharmaceutically and physiologically acceptable excipients. As noted below, a composition according to the present invention can include in addition to an inhibitor compound as disclosed, one or more other molecules of therapeutic use.

The present invention extends in various aspects not only to a substance identified as a modulator of HGF and c-met interaction or activity, property or pathway in accordance with what is disclosed herein, but also a pharmaceutical composition, medicament, drug or other composition comprising such a substance, a method comprising administration of such a composition to a patient, e.g. for anti-cancer, use of such a substance in manufacture of a composition for administration, e.g. for the treatment of acute lung injury, and a method of making a pharmaceutical composition comprising admixing such a substance/composition with a pharmaceutically acceptable excipient, vehicle or carrier, and optionally other ingredients.

A substance/composition according to the present invention such as a promoter of HGF and c-met interaction or binding may be provided for use in a method of treatment.

The invention further provides a method of enhancing or otherwise modulating HGF activity, or other HGF-mediated activity in a cell, which includes administering an agent which enhances the binding of HGF to c-met protein, such a method being useful in treatment of acute lung injury and/or vascular leak. Other disorders related to vascular leak readily suggest themselves to one of ordinary skill in the art.

Whether it is a polypeptide, antibody, peptide, nucleic acid molecule, small molecule, mimetic or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

Pharmaceutical compositions according to the present invention, and for use in accordance with the present invention, may include, in addition to active ingredient, a pharmaceutically acceptable excipient, carrier, buffer, stabilizer or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection, lactated Ringer's injection. Preservatives, stabilizers, buffers, antioxidants and/or other additives may be included, as required.

Examples of techniques and protocols mentioned above can be found in Remington's Pharmaceutical Sciences, 16th edition, Osol, A. (ed), 1980.

The agent may be administered in a localized manner to a lung or other desired site or may be delivered in a manner in which it targets the lungs or other cells.

Targeting therapies may be used to deliver the active agent more specifically to certain types of cell, by the use of targeting systems such as antibody or cell specific ligands. Targeting may be desirable for a variety of reasons, for example if the agent is unacceptably toxic, or if it would otherwise require too high a dosage, or if it would not otherwise be able to enter the target cells.

Instead of administering these agents directly, they may be produced in the target cells by expression from an encoding gene introduced into the cells, e.g. in a viral vector (a variant of the VDEPT technique—see below). The vector may targeted to the specific cells to be treated, or it may contain regulatory elements which are switched on more or less selectively by the target cells.

The agent may be administered in a precursor form, for conversion to the active form by an activating agent produced in, or targeted to, the cells to be treated. This type of approach is sometimes known as ADEPT or VDEPT, the former involving targeting the activating agent to the cells by conjugation to a cell-specific antibody, while the latter involves producing the activating agent, e.g. an enzyme, in a vector by expression from encoding DNA in a viral vector (see for example, EP 0 415 731 A and WO 90/07936).

A composition may be administered alone or in combination with other treatments, either simultaneously or sequentially dependent upon the condition to be treated, such as cancer, virus infection or any other condition in which a HGF mediated effect is desirable.

Nucleic acid according to the present invention, encoding a polypeptide or peptide able to enhance HGF and c-met interaction or binding, or other HGF-mediated cellular pathway or function, may be used in methods of gene therapy, for instance in treatment of individuals with the aim of preventing or curing (wholly or partially) acute lung injury and/or vascular leak.

Vectors such as viral vectors have been used in the prior art to introduce nucleic acid into a wide variety of different target cells. Typically the vectors are exposed to the target cells so that transfection can take place in a sufficient proportion of the cells to provide a useful therapeutic or prophylactic effect from the expression of the desired polypeptide. The transfected nucleic acid may be permanently incorporated into the genome of each of the targeted tumor cells, providing long lasting effect, or alternatively the treatment may have to be repeated periodically.

A variety of vectors, both viral vectors and plasmid vectors, are known in the art, see U.S. Pat. No. 5,252,479 and WO 93/07282. In particular, a number of viruses have been used as gene transfer vectors, including papovaviruses, such as SV40, vaccinia virus, herpesviruses, including HSV and EBV, and retroviruses. Many gene therapy protocols in the prior art have used disabled murine retroviruses.

As an alternative to the use of viral vectors other known methods of introducing nucleic acid into cells includes electroporation, calcium phosphate co-precipitation, mechanical techniques such as microinjection, transfer mediated by liposomes and direct DNA uptake and receptor-mediated DNA transfer.

Receptor-mediated gene transfer, in which the nucleic acid is linked to a protein ligand via polylysine, with the ligand being specific for a receptor present on the surface of the target cells, is an example of a technique for specifically targeting nucleic acid to particular cells.

A polypeptide, peptide or other substance able to interfere with the interaction of the relevant polypeptide, peptide or other substance as disclosed herein, or a nucleic acid molecule encoding a peptidyl such molecule, may be provided in a kit, e.g. sealed in a suitable container which protects its contents from the external environment. Such a kit may include instructions for use.

As described above, the invention provides a method for treating or preventing acute lung injury or vascular leak disorder comprising administering a composition which is capable of enhancing the interaction of HGF and c-met. In particular, the composition enhances the binding of HGF to c-met. Examples of acute lung injury may result from pneumonia or sepsis. It is within the scope of the invention that substances or compositions described in the application can be used individually or in various combinations.

This invention is further illustrated by the following examples, which should not be construed as limiting. The contents of all references, patents, and published patent applications cited throughout this application, as well as the figures, are incorporated herein by this reference.

### EXAMPLES

Materials and Methods

The following materials and methods were used in Examples 1-6.

Reagents

Hepatocyte growth factor (HGF) was purchased from R&D Systems (Minneapolis, Minn.). Anti-phospho-Akt, anti-phospho-GSK3β, and anti-Akt antibodies, as well as LY-294002 were purchased from Cell Signaling (Beverly, Mass.). Anti-GSK3β antisera and Rac activity assay kit were obtained from Upstate Biotechnology (Lake Placid, N.Y.). Anti-PKCα and anti-β-catenin antisera were from Transduction Labs (Lexington, Ky.). Anti-phospho-pan-PKC, anti-pan-ERK, anti-phospho-p44/42 ERK, anti-p38 MAPK, and anti-phospho-p38 MAPK antibodies were purchased from New England Biolabs (Beverly, Mass.). SB-203580, UO126, PP2 and protease inhibitory cocktail were purchased from Calbiochem (La Jolla, Calif.). MLC antibody was produced in rabbits against baculovirus-expressed and purified smooth muscle MLC by Biodesign International (Kennebunk, Me.). Protein G Sepharose 4 Fast Flow was purchased from Amersham Pharmacia Biotech (Piscataway, N.J.). Enhanced chemiluminescent detection system (ECL) was purchased from Amersham (Little Chalfront, Buckinghamshire, England). Reagents used for immunofluorescent staining were purchased from Molecular Probes (Eugene, Oreg.), and all other common reagents were obtained from Sigma Chemical Company (St. Louis, Mo.). NK2 was produced and purified as described in Stahl, S. J. et al. (1997) Biochem. J. 326:763-772.

Cell Culture

Bovine pulmonary artery endothelial cells were purchased from the American Type Culture Collection (ATCC®, Rockville, Md.) and utilized at passage 19-24. Cells were maintained in Medium 199 (Life Technologies, Rockville, Md.) supplemented with 20% (v/v) colostrum-free bovine serum (CFBS) (Irvine Scientific, Santa Ana, Calif.), 15 μg/ml endothelial cell growth supplement (Upstate Biotechnology, Lake Placid, N.Y.), 1% antibiotic and antimycotic, and 0.1 mM non-essential amino acids (Life Technologies). Human pulmonary artery endothelial cells were purchased from Clonetics (Walkersville, Md.), cultured in EBM-2 complete medium (Clonetics) and utilized at passage 5-10. Human alveolar epithelial cells (A549) were purchased from ATCC® and cultured in the same medium as the bovine endothelial cells, except that the endothelial cell growth supplement was omitted. All cells were maintained at 37° C. in a humidified atmosphere of 5% CO₂ and 95% air. Both endothelial cell types grew to contact-inhibited monolayers with the typical cobblestone morphology (Garcia, J. G. N. et al. (1995) J. Cell. Physiol. 163:510-522; Liu, F. et al. (2001) Am. J. Respir. Cell. Mol. Biol. 24:711-719).

Measurement of Transendothelial Monolayer Electrical Resistance

Electrical resistance of EC monolayers was measured using electrical cell impedance sensor system (Applied Biophysics Inc., Troy, N.Y.) as described in Garcia, J. G. N. et al. (2000) J. Appl. Physiol. 89:2333-2343. Cells grown on gold microelectrodes (10⁻³ cm²) in polycarbonate wells act as insulating particles, and the resistance across the monolayers (transendothelial electrical resistance, or TER) is measured in real time. As cells adhere on the microelectrode and intercellular cell contacts are formed or in response to agents which increase junctional integrity, the TER increases (Garcia, J. G. N. et al. (2001) J. Clin. Invest. 108:689-711). In contrast, cell retraction, rounding, or loss of adhesion is reflected by decreases in TER (Garcia, J. G. N. et al. (2000) J. Appl. Physiol. 89:2333-2343). These measurements provide a highly sensitive biophysical assay that indicates the state of cell shape, focal adhesion, and endothelial barrier function (Giaever, I. and Keese, C. R. (1993) Nature 366:591-592; Tiruppathi, C. et al. (1992) Proc. Natl. Acad. Sci. USA 89:7919-7923). All electrical resistance data are presented as normalized values. Briefly, current was applied across the electrodes by a 4000 Hz AC voltage source with an amplitude of 1 V in series with a 1 MΩ resistance to approximate a constant current source (−1 μA). The small gold electrode and the larger counter electrode (1 cm²) were connected to a phase-sensitive lock-in amplifier (5301A; EG&G Instruments Corp, Princeton, N.J.) with a built in differential preamplifier (5316A; EG&G Instruments Corp.). The in-phase and out-of-phase voltages between the electrodes were monitored in real time with the lock-in amplifier and converted to scalar measurements of transendothelial impedance, of which resistance was the primary focus. Transendothelial electrical resistance was monitored for 30 min to establish a baseline resistance (Rₒ) which, for human lung endothelium, was typically between 8 to 12×10³ Ω (wells with R₀<7×10³ Ω or R₀ >15×10³ Ω were rejected). For some experiments, total TER was vectorially resolved into components reflecting resistance to current flow beneath the cell layer (α) and resistance to current flow between adjacent cells (Rb) as described in Garcia et al. (2000) supra utilizing the method of Giaiver and Keese which models the endothelial monolayer mathematically (Giaever, I. and Keese, C. R. (1993) Nature 366:591-592). Thus, changes in α reflect alterations in the net state of cell-matrix adhesion, whereas changes in Rb reflect alterations in the integrity of cell-cell adhesion. TER values from each microelectrode were pooled at discrete time points and plotted versus time as the mean±standard error of the mean (Garcia et al. (2000) supra).

Western Immunoblotting

Endothelial cell monolayers grown to confluence in 12-well plates and challenged with HGF were lysed with 100 μl of 2×SDS sample buffer, and cell lysates were transferred into microcentrifuge tubes and boiled for 5 min. After a brief spin, proteins from 10 μl cell lysates were separated on 12% SDS-PAGE and transferred to nitrocellulose (Schleicher & Schuell, Keene, N.H.) (30V, 18 h). After blocking with PBST (PBS with 0.1% Tween 20) containing 5% non-fat milk for 1 hr, nitrocellulose blots were reacted with primary antibodies diluted in PBST containing 5% BSA for 1 hr, washed with PBST (3×10 min), incubated with peroxidase-conjugated secondary antibodies (goat anti-rabbit IgG, 1:10,000 dilution, Sigma; or goat anti-mouse IgG, 1:10,000 dilution, Bio-Rad Labs, Richmond, Calif.) diluted in PBST with 5% non-fat milk for 1 hr and again washed with PBST (3×10 min). Finally, immunoreactive proteins were detected using ECL. The relative intensities of the protein bands were quantified by scanning densitometry.

Differential Detergent Fractionation of Subcellular Components

Endothelial cells were fractionated into cytosolic, membrane, and nuclear/cytoskeleton fractions as described in Borbiev, T. et al. (2001) Am. J. Physiol. Lung Cell. Mol. Physiol. 280:L983-L990. Briefly, endothelial monolayers were incubated with cytosolic buffer (0.01% digitonin, 10 MgCl₂, mM PIPES, pH 6.8, 300 mM sucrose, 100 mM NaCl, 3 mM MgCl₂, 5 mM EDTA, 5 μM phallacidin) and protease inhibitory cocktail with agitation for 10 min at 40° C. The digitonin-soluble fraction (the cytosolic fraction) was collected, and the residual material was incubated with membrane buffer (0.5% Triton X-100, 10 mM PIPES, pH 7.4, 300 mM sucrose, 100 mM NaCl, 3 mM EDTA, 5 μM phallacidin and protease inhibitory cocktail) with agitation for 20 min at 40° C. The Triton-soluble (membrane) fraction was collected, and the material remaining on the dishes was scraped in SDS buffer (0.5% Triton X-100, 0.5% SDS, 10 mM Tris-HCl, pH 6.8, and protease inhibitory cocktail), sonicated, boiled, centrifuged, and the supernatants (cytoskeletal fraction) together with the other two fractions subjected to SDS-PAGE and western immunoblotting.

Measurement of Rac GTPase Activity

Rac GTPase activity was assessed as described in Garcia, J. G. N. et al. (2001) J. Clin. Invest 108:689-711). Endothelial cells grown in 100 mm dishes were incubated with agonists in serum-free M199. Cells were lysed in 500 μl Mg²⁺ lysis buffer (Upstate Biotechnology, Lake Placid, N.Y.) and homogenized by pipetting. After a brief centrifugation to remove the cell debris, 300 μl of supernatant was incubated with the agarose-conjugated p21-binding domain (PBD) of human PAK-1 (10 μg, 30 min, Upstate Biotechnology). The agarose beads were washed with 1 ml of lysis buffer 5 times and re-suspended in 30 μl of 2×SDS buffer. After 10 min centrifugation at 14,000 g, 15 μl of supernatant from each sample was subjected to electrophoresis in 15% PAGE. After Western transfer, active Rac was detected using an anti-Rac monoclonal antibody. For total Rac protein measurement, 5 μl of the original cell lysates were used for electrophoresis and western analysis.

Immunofluorescent Microscopy

Endothelial cell monolayers grown on gelatinized cover slips were rinsed with M199 and incubated with agonists in the same medium in a 37° C. incubator (5% CO₂). Monolayers were then rinsed with PBS (3×2 min), fixed in 4% paraformaldehyde for 10 min, again rinsed with PBS (3×2 min), and permeabilized with 0.25% Triton X-100 for 5 min. Cells were then washed briefly with PBS (3×2 min), blocked with PBS containing 2% BSA for 30 min and incubated with 1 Unit/mL of Texas Red-X phalloidin (Molecular Probes, Eugene, Oreg.), β-catenin antibody (Transduction Laboratories, Lexington, Ky.), glycogen synthase kinase-3 β (GSK-3β) (Santa Cruz Biotechnology, Inc., Santa Cruz, Calif.), or mono-phosphorylated MLC antisera (see Garcia, J. G. N. et al. (2001) J. Clin. Invest. 108:689-711) for 1 hr. After washing with PBS (3×2 min), cover slips were mounted on slides using SlowFade mounting medium (Molecular Probes). Cells were analyzed using a 60× oil objective on a Nikon Eclipse TE 300 microscope. Images were captured by Sony Digital Photo camera DKC 5000. The same exposure time was applied to all samples within one experiment.

### Example 1

### HGF Increases Trans-Endothelial Electrical Resistance (TER)

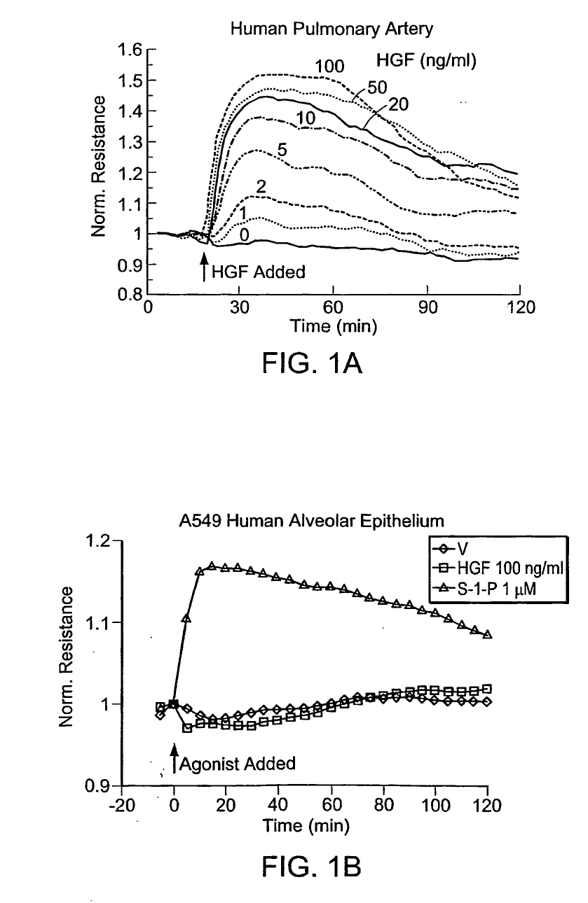

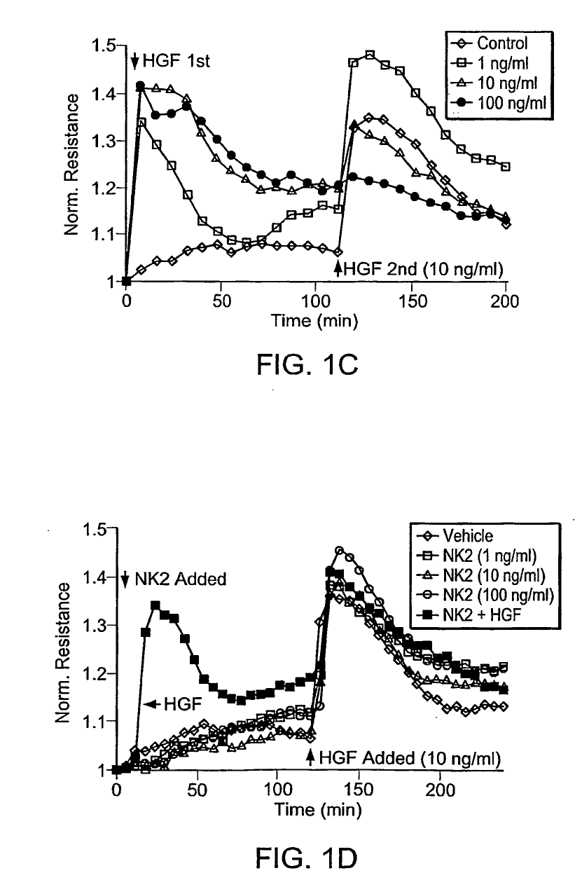

Human and bovine pulmonary artery endothelial cell monolayers, grown on gold microelectrodes to monitor real time electrical resistance (TER), were challenged with serial doses of HGF (from 2 to 100 ng/ml). HGF increased TER in a dose-dependent manner, consistent with barrier enhancement with an elevation in TER clearly evident after 1-2 min (FIG. 1A). HGF-induced increases in TER peaked 15-20 min after exposure to 100 ng/ml of HGF with an increase in resistance from ˜1600 Ω (baseline TER) to ˜2400 Ω, reflecting ˜50% enhancement in barrier function which was sustained above baseline values for several hours. No further significant increases in TER were observed with concentrations of HGF >100 ng/ml. HGF mediated significant elevations in TER across bovine pulmonary artery endothelial cells that was similar in time and concentration dependence to the human cells (Table 1).

TABLE 1
Effect of Angiogenic Factors on Transendothelial Electrical
Resistance across Bovine Pulmonary Artery Endothelium

| | *changes in TER | *changes in TER | *changes in TER |
|---|---|---|---|
| AGENT | 10 min | 30 min | 60 min |
| VEGF | +30 ± 4% | 0% ± 3% | −20 ± 4% |
| Sph 1-P | +50 ± 5% | +50 ± 6% | +35 ± 4% |
| HGF | +25 ± 3% | +35 ± 5% | +35 ± 4% |
| Thrombin | +30 ± 3% | −50 ± 4% | −14 ± 4% |

In these experiments, bovine pulmonary artery endothelium grown on gold microelectrodes for TER measurements were challenged with various angiogenic factors, including VEGF (100 ng/ml), Sph 1-P (1 μM), and HGF (20 ng/ml). For comparison, the edemagenic agent, thrombin (Schaphorst, K. L. et al. (1997) Am. J. Resp. Cell. Mol. Biol. 17:441-455) (100 μM) was added. TER values were compared to vehicle-treated wells at 10 min, 30 min, and 60 min time points. VEGF produces an early increase in TER, which then falls to produce mild barrier dysfunction. Both Sph 1-P and HGF induce a brisk and sustained increase in TER consistent with barrier enhancement. Thrombin rapidly decreases TER values which begin to abate after 30 min (n=at least 5 separate determinations). Change in TER is obtained by calculating the difference between vehicle control TER values and the agonist-mediated TER value at each time point±SEM. The (+) or (−) depicts whether the TER values increased (+) or decreased (−) after agent stimulation.

HGF-mediated barrier protection, however, appears to be specific for lung endothelial cells since HGF did not alter TER values in an immortalized A549 human alveolar epithelial cell line. In contrast, another recently described barrier-enhancing angiogenic agent, sphingosine 1-phosphate (Garcia, J. G. N. et al. (2001) J. Clin. Invest. 108:689-711) did enhance epithelial cell integrity (FIG. 1B), consistent with tissue-and stimulus-specific TER responses.

HGF is known to signal through its specific tyrosine kinase receptor, c-Met. Consistent with this notion, HGF stimulation produced dose-dependent attenuation of TER values in response to subsequent HGF challenge (FIG. 1C), findings consistent with receptor desensitization. HGF/NK2 is a naturally occurring 28 kD truncated HGF isoform derived from an alternatively spliced HGF transcript which in specific cellular systems binds c-Met with high affinity by functioning as a partial agonist (Hartmann, G. et al. (1992) Proc. Natl. Acad. Sci. USA 89(23): 11574-11578). Conversely, NK2 is capable of functionally antagonizing HGF effects on HGF-induced mitogenesis (Guerin, C. et al. (2000) Biochem. Biophys. Res. Comm. 273:287-293; Chan, A. M. et al. (1991) Science 254(5036):1382-1385; Day, R. M. et al. (1999) Oncogene 18:3399-3406). FIG. 1D depicts the complete lack of direct response of human endothelium to NK2 (1-100 ng/ml). Furthermore, subsequent HGF challenge in NK2-pretreated endothelium resembled the effect of HGF in vehicle-treated monolayers, suggesting that the barrier protective response of HGF is not affected by its truncated splice variant.

### Example 2

### HGF Enhances Cortical Actin Ring Formation: Role of RAC GTPases

EC barrier regulation has been shown to be critically dependent upon the dynamics of EC actin cytoskeleton organization (Garcia, J. G. N. et al. (1995) J. Cell. Physiol. 163:510-522; Dudek, S. M. and Garcia, J. G. N. (2001) J. Appl. Physiol. 91:1487-1500). The effect of HGF on the spatial localization of polymerized actin in human endothelial monolayers was investigated by immunofluorescent microscopy. Cells were treated with either vehicle or HGF (100 ng/mL for 5 min). F-actin staining was assessed with Texas red phalloidin and myosin light chain staining evaluated with anti-monophosphorylated myosin light chain polyclonal antibody. Consistent with the evoked increases in endothelial cell TER, HGF (20 ng/ml) produced rapid enhancement of F-actin staining spatially confined to the cortical cytoskeletal ring with reduction of F-actin staining and reproducible increases in mono-phosphorylated myosin light in the same distribution, results similar to those previously noted with the barrier enhancement induced by sphingosine 1-phosphate (Garcia, J. G. N. et al. (2001) J. Clin. Invest. 108:689-711).

In many cell systems, cytoskeletal rearrangements are tightly regulated by Rac GTPases, signaling effectors whose activities are intimately involved with dramatic alterations in the endothelial cortical cytoskeleton and cytoplasmic stress fibers, as has been recently shown (Garcia, J. G. N. et al. (2001) J. Clin. Invest. 108:689-711). Consistent with Rac GTPase mediated cytoskeletal rearrangement, both HGF and Sph 1-P produce rapid (1 min) Rac GTPase activation as determined by p21 Rac-binding domain assay. Pulmonary artery endothelial cells were incubated with HGF (10 ng/mL) or Sph-1-P (1 μM) for 1 or 5 min. Cells were lysed, supernatants collected, and activated GTP-bound Rac was precipitated by agarose-conjugated human PAK-1 p21-binding domain and subsequently immunoblotted by anti-Rac mAb. Total Rac protein was detected using cell lysates. Both Sph-1-P and HGF rapidly and transiently increases Rac activity in endothelial cells.

### Example 3

### HGF-Mediated Endothelial TER Enhancement Involves Phosphatidylinositol 3′ (PI-3′) Kinase Activity

To identify key signaling mediators involved in HGF-induced barrier protection, the role of PI-3′-kinase in HGF-stimulated barrier improvement was examined by measuring HGF-dependent phosphorylation of the serine/threonine kinase, Akt, a well-accepted method of defining PI-3′ kinase activity. Cell homogenates were analyzed by western immunoblotting with anti-phospho-Akt^{ser73} antibody. When endothelial monolayers were incubated with HGF (20 ng/ml) for increasing amounts of time (1, 2, 5, 10, 15, 20, 30, 60, or 120 minutes), AKT is rapidly phosphorylated beginning at 2 min with maximal effect plateauing by 5-30 min. When the cells are incubated for 15 minutes with increasing concentrations of HGF (0.5, 1, 2, 5, 10, 20, 50 or 100 ng/ml), activation was sustained for up to 2 hrs in response to concentrations as low as 5 ng/ml. Pre-treatment with the highly specific PI-3′ kinase inhibitor, LY294002 (25 μM, 30-60 min), abolished HGF-mediated Akt phosphorylation even at HGF concentrations of up to 100 ng/ml, confirming that PI-3′ kinase activity is the key effector in this response.

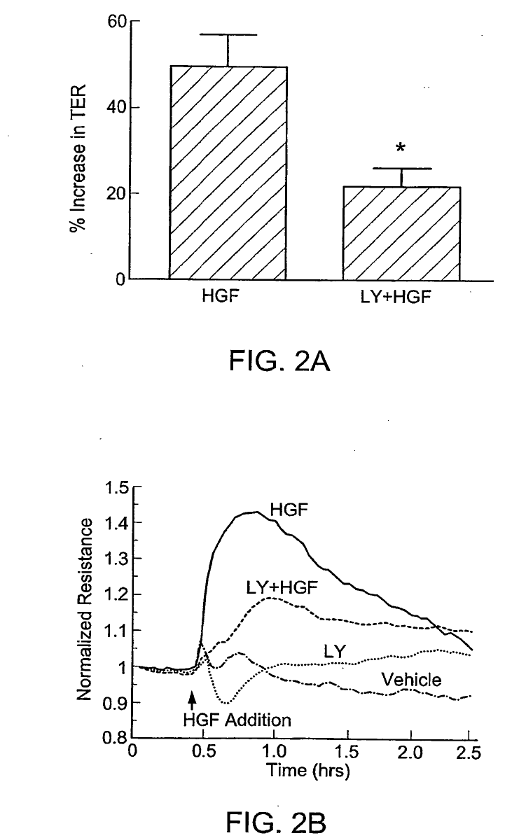

In the next series of experiments, human endothelial cell monolayers were grown on gold microelectrodes and pre-incubated with LY294002, followed by stimulation with HGF (20 ng/ml). PI-3′ kinase inhibition with LY294002 reduced the elevation of HGF-induced TER by >50% (FIGS. 2A and 2B). This fmding represents a fundamental difference between HGF and sphingosine 1-phosphate (Sph 1-P). Sph 1-P, which ligates G protein-coupled Edg receptors, does not require PI-3′ kinase for either endothelial cell migration (Liu, F. et al. (2001) Am. J. Respir. Cell. Mol. Biol. 24:711-719) or barrier enhancement (Garcia, J. G. N. et al. (2001) J. Clin. Invest. 108:689-711). Similarly, LY294002 pretreatment (25 μM, 1 hr) abolished the enhanced cortical actin ring formation elicited by HGF (20 ng/ml, 15 min), but it did not affect Sph 1-P-induced actin reorganization. These results suggest that PI-3′ kinase plays a critical role in the HGF-mediated signaling pathway leading to endothelial cell cytoskeleton reorganization and subsequent barrier enhancement.

### Example 4

### Mitogen-Activated Protein Kinases are Involved in HGF-Stimulated Endothelial Cell Barrier Enhancement

The MAP family of kinases (ERK1/2 and p38) are actively involved in agonist-induced endothelial cell actin reorganization and barrier regulation (Verin, A. D. et al. (2000) Am. J. Physiol: Lung Cell Molec. Phys. 279:L360-L370, 2000; Garcia, J. G. N. et al. (2001) J. Clin. Invest. 108:689-711) and have been noted to participate in HGF-mediated cell activation (Liang, C. C. and Chen, H. C. (2001) J. Biol. Chem. 276:21146-21152). These reports were confirmed in human endothelial cell monolayers incubated with HGF (20 ng/ml, for 1, 2, 5, 10, 15, 20, 30, 60, or 120 min) where activation of p42/44 ERK and p38 MAPK by HGF was detected by immunoblotting with antibodies that only recognize the phosphorylated (activated) forms of ERK or p38 MAPK. The activation of ERKs was evident at 5 min, maximal after 10-15 min with a gradual decline thereafter, but remaining sustained above basal levels for more than 2 hrs. Pretreatment with the specific ERK kinase, MEK, inhibitor UO126 (10 μM, 30 min) completely abolished HGF-stimulated (20 ng/ml, 15 min) ERK activity. The onset of HGF-mediated activation of p38 MAPK was similar to ERK, with plateau at 10-15 min. However, the duration of this response was much more truncated than ERK activation, beginning to decline by 20 min and returning to basal value by 1 hr.

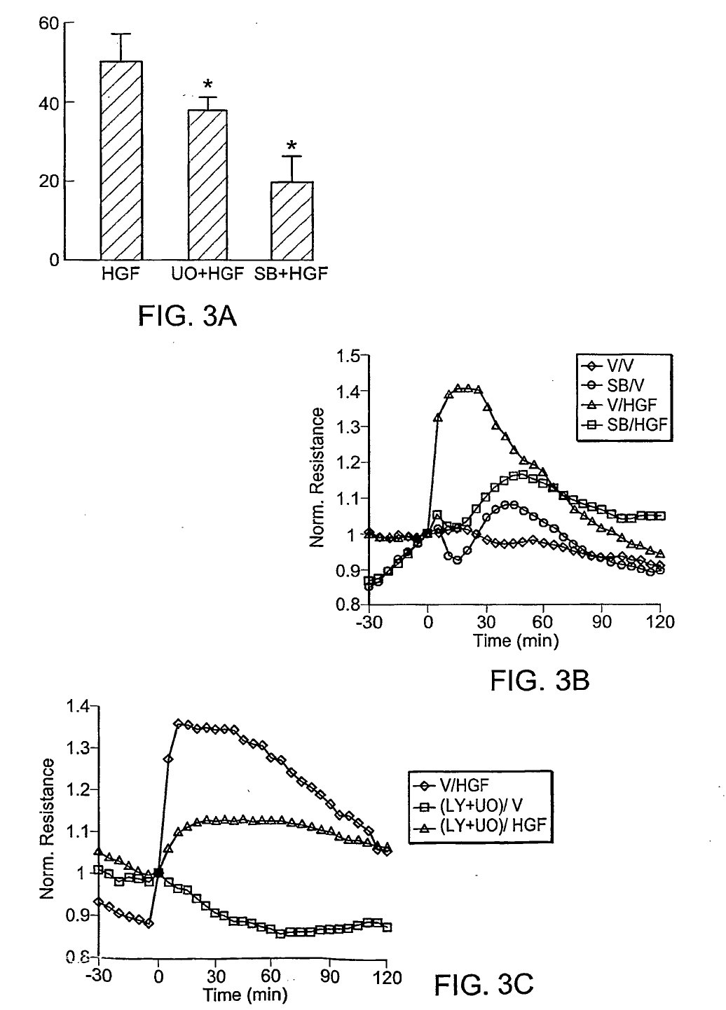

To determine whether MAPK signaling events were important in the barrier enhancement mediated by HGF, endothelial cell monolayers were pre-incubated with UO126 or the p38 MAP kinase inhibitor SB203580 (20 μM, 30 min), followed by stimulation with HGF (20 ng/ml) or Sph 1-P, again used as a negative control (Garcia, J. G. N. et al. (2001) J. Clin. Invest. 108:689-711; Liu, F. et al. (2001) Am. J. Respir. Cell. Mol. Biol. 24:711-719). HGF-mediated barrier protection (FIGS. 3A and 3B) and actin cytoskeletal remodeling were significantly attenuated by p38 MAPK inhibition. Attenuation of HGF-induced TER increases occurred to a lesser extent with MEK inhibition; however, consistent with FIG. 2B, the co-administration of UO126 and LY294002 essentially abolished the HGF-induced increases in TER (FIG. 3C) and together indicate important roles for both p38 MAP kinase and ERK signaling pathways in HGF-mediated endothelial cell barrier protection.

### Example 5

### Protein Kinase C (PKC) Activity is Required for the Enhancement of Endothelial Cell Barrier Function Evoked by HGF

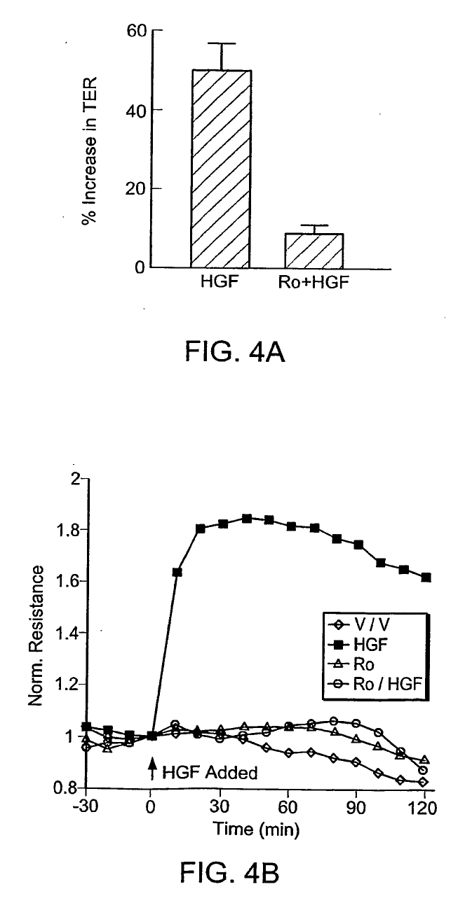

PKC isotype-specific regulation of EC barrier function which evolves in agonist-specific manner has been observed previously (Garcia, J. G. N. et al. (1995) J. Cell. Physiol. 163:510-522; Harrington, E. O. et al. (1997) J. Biol. Chem. 272(11):7390-7397; Schaphorst, K. L. et al. (1997) Am. J. Resp. Cell. Mol. Biol. 17:441-455; Verin, A. D. et al. (2000) Am. J. Physiol: Lung Cell Molec. Phys. 279:L360-L370, 2000). As HGF stimulates PKC activity in certain cell types (Machide, M. et al. (1998) J. Neurochem. 71(2):592-602), the question of whether PKCα is involved in HGF-mediated TER increases in human endothelium was examined. Endothelial cell lysates were blotted with antisera immunoreactive with phosphorylated PKCα, an index of enzymatic activation. Initial experiments confirmed HGF-mediated PKCα activation (detectable at 15 min) detailed by increases in phospho-PKC immunoreactivity, as well as rapid (5 min) translocation to the membrane fraction after HGF. Endothelial cell monolayers were next pretreated with a highly specific pan-PKC inhibitor, Ro-31-2880 (10 μM, 30 min), which preferentially inhibits membrane-bound PKC isoforms. As shown in FIG. 4A, treatment with Ro-31-2880 produced an 80% reduction in HGF (20 ng/ml)-evoked increases in TER, implying a major role for PKC in barrier enhancement mediated by HGF.

### Example 6

### Role of Glycogen Synthase Kinase 3β (GSK3β) In HGF-Induced Endothelial Barrier Enhancement

Increases in barrier function may be conceptualized as reflecting either enhanced cell-matrix adhesion via focal adhesions or strong increases in cell-cell tethering produced by homotypic cadherin linkage via catenins to the actin cytoskeleton (Dudek, S. M. and Garcia, J. G. N. (2001) J. Appl. Physiol. 91:1487-1500). For example, β-catenin is a critical component of the adherens junction (Dejana, E. et al. (1997) Ann. NY Acad. Sci. 811:36-43) and essential to endothelial cell monolayer integrity and paracellular barrier regulation (Dudek, S. M. and Garcia, J. G. N. (2001) J. Appl. Physiol. 91:1487-1500). Increased β-catenin availability has been postulated to increase intercellular tethering, and thus enhances cell-cell adhesion (Hinck, L. et al. (1994) J. Cell. Biol. 124(5):729-74 1). Consistent with this cell-cell tethering paradigm, partitioning of electrical resistance vectors across human endothelial cells grown on gold microelectrodes identified sharp increases in paracellular junction resistance (Rb) after HGF treatment, results which were identical to the TER vectorial-derived responses to Sph 1-P (Garcia, J. G. N. et al. (2001) J. Clin. Invest. 108:689-711). Consistent with enhanced paracellular resistance, HGF-stimulated human endothelial cells examined by immunofluorescent microscopy demonstrate increased β-catenin immunoreactivity along cell borders with co-localization with the cortical cytoskeleton. These two events were dependent upon PI-3′ kinase activation as LY294002 diminished this response. Differential detergent fractionation revealed enhanced β-catenin and VE cadherin partitioning to the Triton-insoluble cytoskeletal fraction, and immunoprecipitation of β-catenin after HGF challenge showed enhanced association with VE cadherin, results which indicate increased tethering of the cytoskeleton to zonula adherens proteins.

HGF has been reported to increase the phosphorylation status of GSK3β, a multi-functional enzyme involved in glycogen synthesis and protein synthesis regulation activity in mammary epithelial cells (Papkoff, J. and Aikawa, M. (1998) Biochem. Biophys. Res. Commun. 247:851-858). Of potential importance to barrier regulation, GSK3β phosphorylation results in enzymatic inactivation and increases the level of uncomplexed cellular β-catenin (Papkoff, J. (1997) J. Biol. Chem. 272(7):4536-4543). GSK3β phosphorylation can be catalyzed by multiple pathways including the PI-3′-kinase target Akt kinase (Cross, D. A. et al. (1995) Nature 378(6559):785-789), the p38 MAP kinase-activated protein kinase 1 (MAPKAP-K1), MEK-dependent pathways (Sutherland, C. et al. (1993) Biochem. J. 296:15-19), or PKC (Isagawa, T. et al. (2000) Biochem Biophys. Res. Comm. 273:209-212). Given that the results presented herein indicate that each of these signaling paradigms is involved in HGF-stimulated barrier enhancement, the HGF mediated phosphorylation status of GSK3β in human endothelial cells was examined by Western immunoblot analysis with antisera specific for the phosphorylated N-terminal Ser⁹ of this enzyme (Sutherland, C. et al. (1993) Biochem. J. 296:15-19). Concomitant with HGF-induced TER augmentation, increases in phosphorylation of GSK3β after HGF treatment was detected by increased immunofluorescence and prominent by Western blotting at 2 min, with peak intensity leveling off at 15-30 min, although remaining above basal levels for more than 2 hr. Phosphorylation of GSK3β induced by HGF was dramatically attenuated by pharmacologic inhibitors of PI-3′ kinase, ERK and p38 MAPK, and PKC.

### Example 7

### Use of HGF for the Treatment of Acute Lung Injury

The experiments described below are designed to guide clinical trials using HGF receptor agonists for the treatment of sepsis or pneumonia related acute lung injury and to understand the mechanisms by which these therapies may attenuate the lung injury.

Murine Studies

Eight to ten week-old C57BL/6 mice weighing approximately 25-30 grams are anesthetized with a 0.03 ml intraperitoneal injection of 10:1 ketamine (100 mg/ml or 135 mg/kg) and acepromozazine (10 mg/ml or 1.5 mg/kg), with additional anesthetic administered as necessary. Proper anesthesia is verified by paw and tail pinching. Tracheostomy is performed with a one-inch long 20-gauge catheter via a midline neck incision (Johnson and Johnson). LPS (1.5 mg/kg diluted into 100 μl PBS or saline) or vehicle (PBS or saline) is then introduced into the trachea. The tracheostomy catheter is removed, the neck incision closed, and the animals are allowed to recover for 24 hours with free access to water and chow. Subsequently, animals are anesthetized as described above. Tracheostomy is placed as detailed above. Two hours of mechanical ventilation (Harvard Apparatus, Boston, Mass.) are then commenced by one of two strategies. Lung protective ventilation (CV_{LP})is performed with low tidal volume (6-8 cc/kg) combined with PEEP set at 3 cm H₂O. Non-lung protective ventilation (CV_{NLP}) proceeds with high tidal volume (12-17 cc/kg) and 3 cm H₂O PEEP. The rate will be prescribed to maintain consistent minute ventilation between groups (250 breaths per minute and 125 bpm respectively). Inspired oxygen (FᵢO₂) is set to room air (21%).

HGF Dosing (n=10): Doses of 0.1-10 mg/kg (Suzuki, S. (1999) Transplant Proc. 31:2779-82) are used. Optimal dosing is determined by a dose-escalation protocol, starting at 0.25 mg/kg in the first animal. HGF is escalated with each animal by 0.25 mg/kg until a significant decrease in Evan's blue extravasation relative to control animals is observed. The elimination half-life of HGF is determined after intravenous administration (Troncoso, P. and Kahan, B. D. (1998) Clin. Biochem. 31:369-73). A single administration should be adequate to observe the desired effect. In this model, mice are challenged with LPS for 24 hrs and then placed on mechanical ventilation for 2 hrs above.

Canine Studies

Lung Injury Models:

Endotoxin: Dogs require higher doses of LPS to induce lung injury than other species (Brigham, K. L. and Meyrick, B. (1986) Am. Rev. Respir. Dis. 133:913-27). A model of endotoxin-induced acute lung injury achieved through intravenous infusion of E. coli lipopolysaccharide (LPS, 055:B5 Sigma Catalog No. L4005) at 0.75 mg/kg/hr over 4-6 hours administered through a right atrial catheter is used. Lung injury has been quantified by a 50% decrease in PₐO₂/FᵢO₂ ratio and an increase in lung wet-weight to dry-weight ratio. Edema accumulation is most pronounced in dependent lung regions. In order to optimize consistency with the murine model, a canine model of lung injury induced by intratracheal instillation of endotoxin is used. Initially, LPS 5 mg/kg is introduced by bronchoscopic injection of four aliquots of 25 cc, 4-16 hours prior to mechanical ventilation. LPS is distributed to different regions of the lung between aliquots through postural manipulation by protocol. Supportive care and continuous monitoring is provided for the duration of six hours as previously described.

Saline lavage (SL) lung injury is induced with normal saline warmed to 38° C. instilled at a dose of 40 ml/kg via the endotracheal tube from a height of 60 cm for a maximum dwell time not to exceed 120 sec. Following dwell, the lavage is allowed to drain by gravity. The process is repeated, following a ten-minute recovery period, until the PₐO2 remains below 125 mm Hg for 10 minutes. The animal is changed between prone, supine, and right and left lateral positions between each lavage. A minimum of four washes will be performed; typically 6-8 are required.

Oleic acid (OA) lung injury is induced by infusion of 0.08 ml/kg oleic acid dissolved in absolute alcohol into a central line or PA catheter over 20 minutes. The animal is positioned prone for the first 10 minutes of the infusion and then turned supine for the remainder.

Mechanical ventilation: In general, dogs require larger tidal volumes than humans on a per kilogram basis (Venegas, J. G. et al. (1986) J. Appl. Physiol. 60:1025-30). As a result, the lung protective strategy, CV_{LP}, employs tidal volumes of 8-10 cc/kg and 8-10 cm H₂O end-expiratory pressure (PEEP). The non-lung protective ventilation strategy, CV_{NLP}, utilizes 15-18 cc/kg tidal volumes and zero PEEP. Once tidal volume is set, respiratory rate is adjusted to maintain a pH>7.20. FᵢO₂ of 0.30 will be used and increased as required to maintain SₚO₂>88% or PₐO₂ above 60 mm Hg.

HGF dose determination: HGF is initiated at a dose of 5 mg/kg injected one hour into endotoxin infusion. Subsequent animals are treated with escalating doses in 2.5 mg/kg increments administered one hour into endotoxin infusion until the rise in extra-vascular lung water (EVLW) is reliably attenuated by 50% or more or until cardio-suppressive side effects prohibit further escalation (Kutzsche, S. et al. (2001) Crit Care Med. 29:2371-3276; Guo, J. et al. (1999) Pflugers Arch. 438:642-8).

Experimental endpoints: The animal is followed for 6 hours after initiation of endotoxin infusion. To characterize the injury and the response to therapy, measurements of hemodynamics (blood pressure, central venous pressure, pulmonary artery pressure, pulmonary capillary wedge pressure, and cardiac output), gas exchange (arterial and venous blood gases, shunt fraction, and dead space fraction), extravascular lung water (EVLW), and lung mechanics (peak airway pressure, pleural pressure, end-expiratory pressure) are monitored hourly throughout the protocol. Serum samples are drawn hourly and BAL is performed prior to endotoxin, midway through the study, and just prior to conclusion. Pulmonary extravasation of Evan's blue dye over the final 30 minutes of the study is measured. After sacrifice, the chest is opened and 10 regional lung tissue samples taken and processed for genomic microarray analysis, Evan's blue concentration determination, histologic scoring, and wet-to-dry lung weight ratios. Serum and BAL samples are assayed for standard biomarkers, currently including inflammatory mediators (TNFα, IL-1β, IL-6, IL-8), nitrotyrosine proteins, Von Willebrand Factor, sphingosine-1-phosphate, surfactant proteins A and B, isoprostanes, anti-oxidants, protein concentration, and novel biomarkers to be developed.

Equivalents

Those skilled in the art will recognize, or be able to ascertain using no more than routine experimentation, many equivalents to the specific embodiments of the invention described herein. Such equivalents are intended to be encompassed by the following claims.

1. A method for reducing vascular leak, comprising administering to mammalian vascular endothelial cells an effective amount of HGF or functional derivative thereof.

2. The method of claim 1, wherein the cells are lung cells.

3. The method of claim 1, wherein the cells have been identified and selected for treatment to reduce vascular leak and the HGF or functional derivative thereof are then administered to the identified and selected cells.

4. The method of claim 1, wherein the HGF or functional derivative thereof increases transendothelial electrical resistance (TER) by at least about 10 percent.

5. The method of claim 1, wherein administration of the HGF or functional derivative thereof increases the activity of one or more of the PI-3′ kinase pathway, mitogen-activated protein kinases, and protein kinase C induced by activation of the c-met receptor.

6. The method of claim 1, wherein administration of the HGF or functional derivative thereof inhibits the activity of GSK-3β induced by activation of the c-met receptor.

7. The method of claim 1, wherein the cells are human cells.

8. The method of any one of claims 1 through 7, further comprising administering an effective amount of sphinogsine 1-phosphate to the cells.

9. A method for treating a mammal suffering from a disease or disorder associated with vascular leak, comprising identifying and selecting the mammal on the basis of the disease or disorder, and administering to the mammal an effective amount of HGF or functional derivative thereof.

10. The method of claim 9, wherein the disease or disorder is associated with vascular leak induced or resulting from acute lung injury.

11. The method of claim 9, wherein the mammal is suffering from pneumonia.

12. The method of claim 9, wherein the mammal is suffering from sepsis.

13. The method of claim 9, wherein the disease or disorder is selected from the group consisting of: trauma, inflammation, infection, pulmonary aspiration of stomach contents, pulmonary aspiration of water, near drowning, burns, inhalation of noxious fumes, fat embolism, blood transfusion, amniotic fluid embolism, air embolism, preeclampsia, eclampsia, vascular leak syndrome, edema, organ failure, poisoning, and radiation.

14. The method of claim 10, wherein the HGF or functional derivative thereof is administered to the mammal within about 6 hours after the mammal has suffered acute lung injury.

15. The method of claim 10, wherein the HGF or functional derivative thereof is administered to the mammal within about 18 hours after the mammal has suffered acute lung injury.

16. The method of claim 10, wherein the HGF or functional derivative thereof is administered to the mammal within about 1 week after the mammal has suffered acute lung injury.

17. The method of any one of claims 9 through 16, wherein the HGF or functional derivative thereof is administered intravenously.

18. The method of any one of claims 9 through 16, wherein the HGF or functional derivative thereof is administered via bronchoscopic injection.

19. The method of any one of claims 9 through 16, wherein the HGF or functional derivative thereof increases transendothelial electrical resistance (TER) by at least about 20 percent.

20. The method of any one of claims 9 through 16, wherein the mammal is a human.

21. The method of claim 9, wherein administration of the HGF or functional derivative thereof increases the activity of one or more of the PI-3′ kinase pathway, mitogen-activated protein kinases, and protein kinase C induced by activation of the c-met receptor.

22. The method of claim 9, wherein administration of the HGF or functional derivative thereof inhibits the activity of GSK-3β induced by activation of the c-met receptor.

23. The method of any one of claims 9 through 16, further comprising administration of an effective amount of sphingosine 1-phosphate to the mammal.
